(19) United States
(12) Patent Application Publication
(10) Pub. No.: US 2006/0241336 A1
(43) Pub. Date: Oct. 26, 2006

(54) SYSTEM AND METHOD FOR DREAM THERAPY TO FACILITATE LIFE REVIEW

(76) Inventor: Jonathan David Gerson, Seattle, WA (US)

Correspondence Address: BLACK LOWE & GRAHAM, PLLC, 701 FIFTH AVENUE, SUITE 4800, SEATTLE, WA, 98104, US

(21) Appl. No.: 11/380,069

(22) Filed: Apr. 25, 2006

Related U.S. Application Data
(60) Provisional application No. 60/675,042, filed on Apr. 25, 2005.

(51) Int. Cl. A61M 21/00

(52) U.S. Cl. 600/27

(57) ABSTRACT

A method of dream therapy to facilitate life review is disclosed. In one embodiment, the method may comprise recalling a dream fragment, associating with the dream fragment and amplifying the dream fragment. The method may further comprise of reminiscing life events. The amplified dream fragment and reminisced life events may be combined to conduct a life review. In one particular embodiment, the present invention may be used to counsel seniors at the end-stage of life in self-acceptance and closure.

### PRIORITY CLAIM

This application claims priority and the benefit of the filing date of U.S. Provisional application Ser. No. 60/675,042 entitled METHOD OF DREAM THERAPY TO FACILITATE LIFE REVIEW, filed Apr. 25, 2005 and is incorporated by reference in its entirety herein.

### COPYRIGHT NOTICE

This disclosure is protected under United States and International Copyright Laws. © 2006 Jonathan David Gerson. All Rights Reserved. A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### FIELD OF THE INVENTION

This invention relates generally to a method of dream therapy and, more specifically, dream therapy to facilitate life review.

### BACKGROUND OF THE INVENTION

Everyone in the world dreams. Yet, dreams are perhaps the least understood of any universal function. In its simplest terms, dreams may be described as images. They may be characterized as the mind's attempt to reveal the dreamer's innermost doubts and desires. As Dr. June Singer wrote, “dreams are a self-portrait of the unconscious at a given moment.” Boundaries of the Soul (1994). Dreams are often recalled as fragments, an incomplete picture of the self-portrait. Some may suppress, censor, or otherwise conceal parts or all of their dreams out of embarrassment or fear. Others may simply forget the dream in their waking moments, only recalling bits and pieces. That fragment of dreams, however, may provide an invaluable preview into the mind's psyche.

A plethora of information is available to anyone seeking to determine the “meaning” behind their dreams. Thousands of Internet websites and published literature offer translations of almost every imaginable dream theme from A to Z. These interpretations, however, may be inaccurate, inconsistent and not standardized amongst references. The definition of a dream image on one website's dictionary may not be consonant with the definition provided in a published dream dictionary.

Other techniques like dream analyses, for example, examine dreams to determine their underlying or disguised motivations, symbolic meanings or symbolic representations. In searching to unravel the mysteries of cognition, a dream analyst may attempt to expand the dream fragment into a wider thought. Yet, this wider thought may be limited to remembering past experiences and not embracing circular life events. Dream analysis may be limited in context and may not be able to relate the dream images to commonly existing images that resemble life stages, including, for example, childhood, adolescence, and adulthood.

Dreams are one method of reminiscing and may be characterized as a short movie played out in the theatre of the mind. The film is often not linear but circular, existing without time and space. Reminiscing becomes optionally advantageous at the end stage of life. For instance, an elderly person's experience at a long-term care facility may be very isolating. Residents are provided structured activities such as music and bingo, but their emotional needs may go unmet. Interaction between staff and resident may not provide an opportunity for the patient to adequately express themselves and reminisce. In particular, reminiscing may become an optionally advantageous aspect in treatment and eventually in accepting closure.

### BRIEF DESCRIPTION OF THE DRAWINGS

Preferred and alternative embodiments of the present invention are described in detail below with reference to the following drawing.

FIG. 1 is a block diagrammatic view of a method of dream therapy to facilitate life review according to an embodiment of the present invention;

FIG. 2 shows a method of conducting dream therapy in one embodiment;

FIG. 3 shows a method of performing analysis;

FIG. 4 shows a method of life review; and

FIG. 5 shows a light pen in one embodiment.

### DETAILED DESCRIPTION OF THE PREFERRED EMBODIMENT

An embodiment is directed to a method of dream therapy to facilitate life review. Embodiments of the present invention may provide a counseling approach to construct a thorough inquiry into the significant features of the patient's life. Embodiments may provide a method of summarizing the patient's life using dream images to facilitate closure and self-acceptance.

In one embodiment, a method of dream therapy to facilitate life review comprises recalling a dream fragment, associating with the dream fragment and amplifying the dream fragment. The method may also comprise reminiscing about life events. Finally, the amplified dream fragment and the reminisced life events are combined to conduct a life review.

In one embodiment, a preferable goal is to help facilitate an end of life review for a given client using their own dreams as a catalyst for the reminiscence. The life story is told and listened to by the therapist with the aim of integrating the disparate recollections into a coherent story. How a client tells their life story speaks volumes about themselves as a person. A typical session is a dialogue between the client and the therapist with an emphasis on life's successes and triumphs. A client that is feeling depressed is redirected, in one embodiment, to “re-story” their life in a more optimistic fashion. Enhancement of mood is a major therapeutic goal. The process can break through the isolation and losses of the elderly by creating a bond between the client and therapist. One embodiment is very much a “socialization” therapy. Friendships forged when conducting this therapy are very deep and the process of self-discovery can continue until death. A successful client can end their earthly existence on a happier note than when they commenced. The process of discovering hidden meaning in dreams combined with free association and reminiscing can team up to provide an enhancement in general outlook on life. The client can emerge with a newly found insight and will not feel so isolated in the process of aging and dying.

In one embodiment the following are possible users of the system and method: hospice workers that need a new approach to their work, nursing home staff who need a new attitude with their patients, assisted living facilities' activities staff who are always looking for new activities, conventions on aging who are always looking for speakers and presenters, senior centers who are always looking for something new, private pay clients who can afford to get some extra companionship. In an alternate embodiment, families could give their loved ones a gift certificate of 6 one hour visits for x amount of dollars.

A method of using one embodiment includes: a client elects to have therapy; the client is evaluated to see if he/she is appropriate for the therapy using the above mentioned guidelines; the client sees a short video showing briefly how the process works; the client is instructed how to recall dreams on a daily basis as described in the workshop; the first appointment is set and the client signs paperwork; the first meeting begins with a general assessment of the client's history; the assessment begins with birth and follows through the present day; the client is to tell the family history to the best of their ability, if details are fuzzy, the family can be called in at a later time to fill in crucial details; and a pattern of significance should develop.

In one embodiment the therapist is instructed to look for “pockets of memory.” These are defined as times in the client's life that their recollection is the strongest. Usually these are events that were exceptional. They can be exceptionally positive or negative. The therapist merely records these anecdotes rather than judges them to be significant or insignificant at this time. In one embodiment a therapist is “discovering best moments.” The therapist time is finding the time in the client's life that they were at their functional best and the goal is to get them to reminisce about those best and often buried moments. Finding these buried memories can lead to a conversation that is more rooted in positive experience and therefore more authentic. Clients have something that they still care about, or cared about greatly, the goal is to unbury them and the dream fragment is like a laser beam, it homes in on the central issues in the client's unconscious self.

Next, once the telling of the life story is complete to the client's satisfaction, the dream fragments can be brought in; and a dream fragment is analyzed.

The client should bring into each session any dream material recalled and recorded. This may not always be possible but, an earnest attempt should be made. The dreams should be read out loud by the therapist several times. The client needs to hear the content spoken to them. The fragment is then analyzed for key words or key ideas. A list of these key words is compiled.

In one embodiment, in order to promote free association, the words are then spoken back to the client. For example, if the list is car, boy, and/or money, the therapist should tell the client that I am going to read these words to you and you tell me what comes to mind. 1. Car—“I had a model-T as a young girl.” 2. boy—“I was often called a tomboy as a young girl.” 3. Money—“We never had much money.” So you continue in that fashion until you have exhausted the list.

The next step is to amplify the associations into reminiscence. For example, a conversation might evolve in this fashion. Therapist—“So you grew up on a farm in Walla Walla County and your first car was a model-T. Do you remember driving in the car with your Dad?” Client—“Why yes, I was just a little girl and I loved to ride in the car, we even took the dog with us.” Therapist—“Tell me more about driving in the car with your Dad.” Client—“Well, we used to drive to the church on Sunday and have a picnic in the park.” The conversation should proceed from there. As the therapist learns more about the client a general picture of the client's life emerges. Small recollection should be complimented by the therapist. After 6 to 8 sessions, or when the client and therapist decide to terminate the therapy the process is halted.

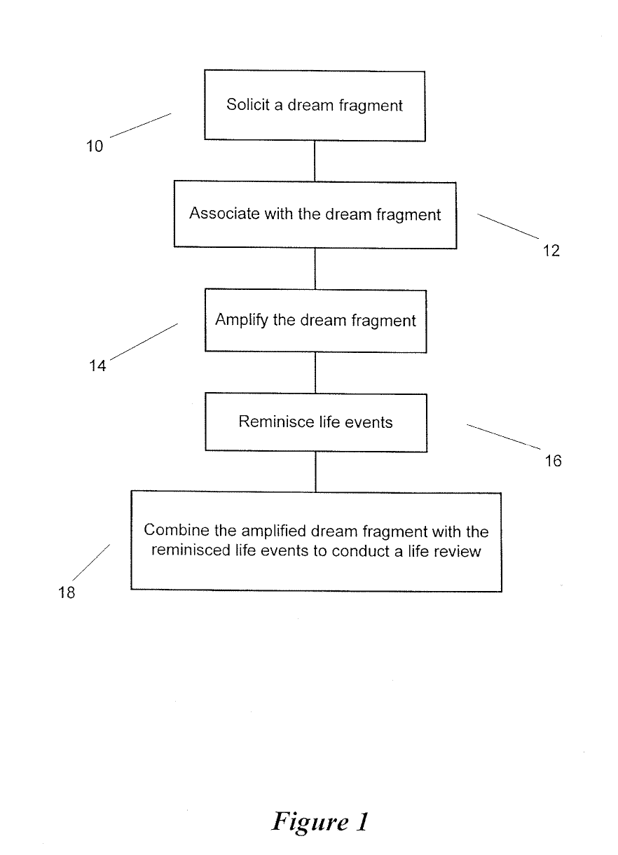

FIG. 1 is a block diagrammatic view of a method of dream therapy to facilitate life review in one embodiment. At a block 10, a dream fragment is solicited from a patient. As the term is used herein, a dream fragment may be a segment of a dream taken at any point during the dream. Alternatively, a dream fragment may be an entire dream sequence. In one particular embodiment, the dream fragment may be recalled by keeping a dream journal. Dream journaling may include a daily, weekly, monthly, or other suitable periodical unit or interval used by the dreamer to chronicle their dreams. Dream journaling may be optionally advantageous when recalling dream fragments that are quickly erased or forgotten. Alternatively, dream journaling may be optionally advantageous in recalling detailed information about the dream fragment.

In one embodiment, dream journaling may include keeping a pen and paper nearby, so that as soon as the dreamer awakes from the dream she can immediately or soon thereafter record the event. In one embodiment a user would use a light pen. A light pen is a pen that when activated puts of just enough light to write. This allows a user to write at night without turning on all the lights. In an alternate embodiment, dream journaling may include journaling in a laptop, desktop or other suitable personal computer. One will appreciate, however, that alternate and suitable journaling methods may exist and apply.

In another embodiment, recalling a dream fragment may include freezing. Freezing refers to freezing in place upon awakening from a dream, i.e. refraining from immediate and sudden movement. Dreams may be easily forgotten as soon as the dreamer awakens from the dream and begins to move around. Freezing may lock the dream in one's memory by keeping it fresh in the mind after awakening until such time the dreamer is able to construct and retain an outline of the dream. In one aspect, freezing may include dream journaling, as described above. The dreamer may write down an outline of the dream immediately after freezing. In another aspect, freezing may include dream journaling in the present tense. “I am riding on the bus.” “I am washing my car.” The present tense may put the dreamer in an active position and keep the dream happening while it is still vital and exciting.

At a block 12, association may be made with the dream fragment. To associate may include bringing something into relationship. In one particular embodiment of the present invention, association may include free association. Free association may include any unconstrained association made between ideas, words, and thoughts. For example, a word may be presented and the reply sought may include the first word that comes to mind. In a sense, free association is much like brainstorming.

In one embodiment, the dream fragment may be associated with by choosing one aspect of the dream and asking the dreamer to associate with that particular aspect. For instance, where the dream comprises of riding a horse on a farm, the dreamer may be asked to associate with the word ‘horse’. The dreamer may be asked what comes to mind when they hear this word. Other suitable inquiries and associations may include emotional and visual associations with the images and other associations that may be triggered.

Associations may be amplified at a block 14. Amplification may include reinforcing the dream image with outside factors, such as those obtained from the association, thereby allowing the image to resonate in the dreamer's consciousness. For example, the dreamer may be asked what they think the chosen word for association means. In one aspect, amplification may lead to reminiscing about life events, at block 16. Examples of reminiscing may include—What does the associated word remind them of? Anything from their past? Anything going on currently? How does this dream fit into the overall context of life now? In one embodiment, questions concerning reminiscences may be asked by open ended questions to permit the patient to speak freely about their past. Examples of open ended questions may include—What were the happiest times of your life? What event(s) in life shaped your character the most?

In one embodiment, reminiscing includes learning a full background history, including, for example, where the patient grew up, whether they were married, how many children they had. Learning the background may assist in putting the dream fragment and reminiscences into context and in comparing the dream fragment with common images already existing that resemble it. As Aristotle once noted, “learning to see resemblances is the beginning of all art in dream work.” R. Bosnak, A Little Course in Dreams (1998).

At a block 18, the amplified dream fragment, block 14, and the reminisced life events, block 16, may be combined to conduct a life review. In one embodiment, life review may include a process of reflection that positions memories and recollections extended and amplified by the dream fragment into appropriate life stages. Examples of life stages may include childhood, adolescence, adulthood, middle-age, and senior adult life. Returning to the horse and the associated terms and reminiscences, for example, the dreamer may reflect upon a time in her life when she rode horses, perhaps her childhood, and remember her parents and siblings and this may bring back fond and happy memories. In one embodiment, life review may provide enhanced mood and ego integrity, as the dreamer reminisces cheerfully about past events. With an enhanced outlook on their past life events, the dreamer may be able to foster an increased awareness and acceptance of their current senior stage and, ultimately, death.

One will recognize however, that not all dream fragments nor all life reviews are pleasant. In an alternate embodiment, nightmares may be recalled and buried unconscious material may be unearthed. Disturbing content and emotional outbreaks may be discussed and noted with full validity. Past devastating experiences, however, may represent an attempt to integrate past trauma. In one aspect, traumatic experiences should be reminisced with sufficiency to promote healing and stasis. In another aspect, traumatic experiences should be reminisced at a minimal and diverted or redirected to “happier” memories. It may be appreciated, however, that although a person's past cannot be altered, and their reaction to the past may be changed with the present invention to achieve resolution.

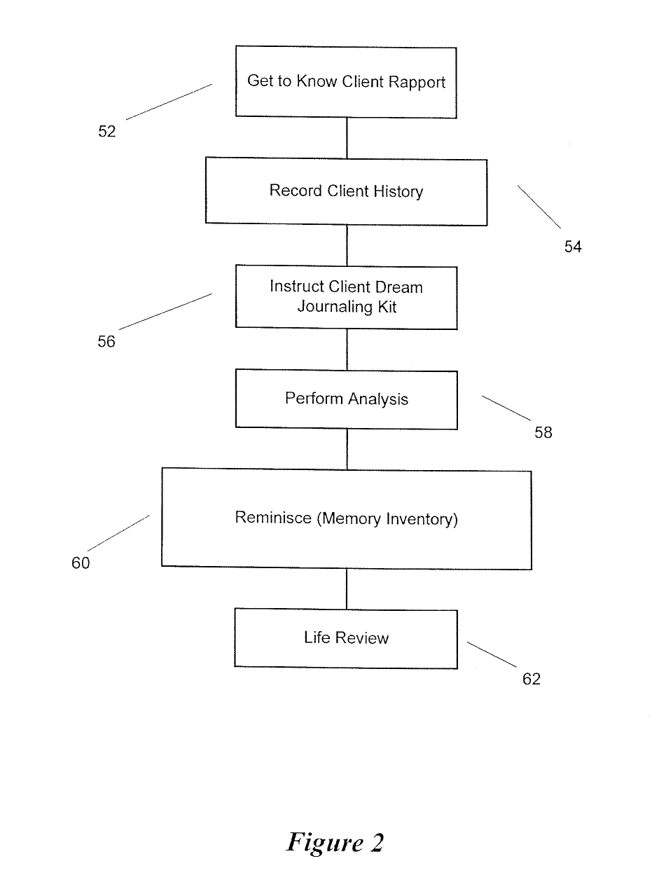

FIG. 2 shows a method of conducting dream therapy in one embodiment. At block 52, a therapist gets to know the client and attempts to build client rapport. The following exercises are advantageous in this method however, are not exhaustive. Exercise #1—Exploration of your own fears/prejudices and associations with the dream process is essential to dream work. Exercise #2—Relaxation of the client is essential for the therapist to have maximum access to the unconscious. i.e. Deep breathing, visualization/meditation allows the therapist to be open to clients in a neutral manner—eliminating personal projections. Exercise #3—Building good client/therapist rapport

At block 54, the therapist will review and record a client's history. In one embodiment the therapist will organize personal history in terms of: memories; eras; relics; parables; lessons; and/or ceremonies.

At block 56, a client is instructed on a client dream journaling kit. At this step in the process a client is taught about motivation, perseverance, patience; self observation; chronicling the inner journey; and how to journal the emotion of the dream

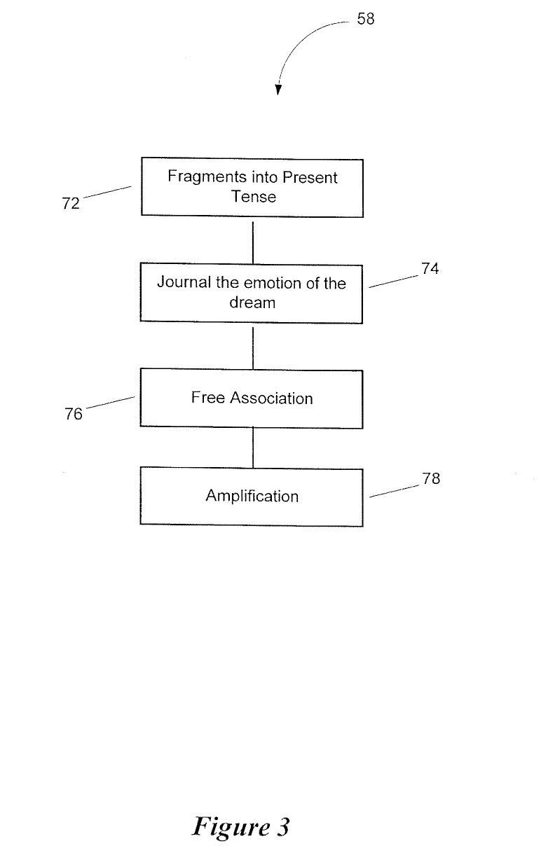

At block 58 analysis is performed. The performance of analysis is further described in FIG. 3.

At block 60 reminiscence (memory inventory) takes place. A therapist will look for “pockets of memory” and relate discovered unconscious material to conscious memory. Then he/she will place the memory into eras; reminisce about the era of the dream fragment and memory. Dreams bring the past alive and the therapist joins the journey. The therapist is searching for the client's “best moments,” and in so doing the therapist bolsters self-esteem by finding a time in the client's life that they can recall with some authority and confidence.

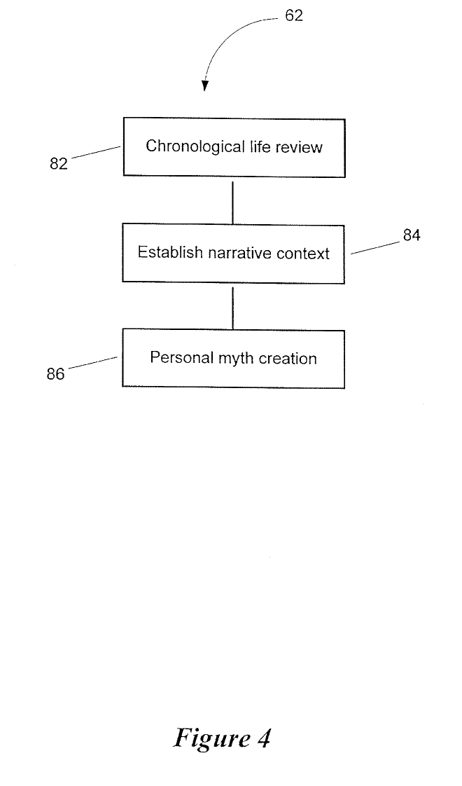

At block 62 a life review is conducted. Life review is further explained in FIG. 4.

FIG. 3 shows a method (58) of performing analysis. At block 72 dream fragments are transformed into present tense. At block 74 a client will journal the emotion of the dream. At block 76 a user will participate in free association. At block 78 a client will participate in dream amplification. Further, and in more detail a client will select a dream fragment. The therapist/analyst will participate in dialogue with patient and take key words in the fragment and free associate to relate to the clients life history. The discussion is expanded by using amplification. The focus is on emotionally charged words where drama is occurring, in one embodiment. The therapist/analyst will discuss and reiterate obstacles or conflicts and paraphrase, mirror emotions and summarize and move ahead to reminiscence

FIG. 4 shows a method (62) of life review. At block 82 there is a chronological life review. At block 84 a narrative context is established. At block 86 a personal myth creation takes place. A question that may be addressed, at this point, is how we move from experience to memory to meaning. In other words “Dream the myth forward.” In one embodiment authentic narrative of the client's everyday life and everyday situations is the foundation of meaning. We all suffer, struggle and have our inner and outer battles. One embodiment synthesizes the forgotten heroic aspects of these battles and moves the client towards self-acceptance and integration. This step includes validating the unique and special aspects of their lives. The described therapy re-stories the client's life experiences into a personal heroic myth.

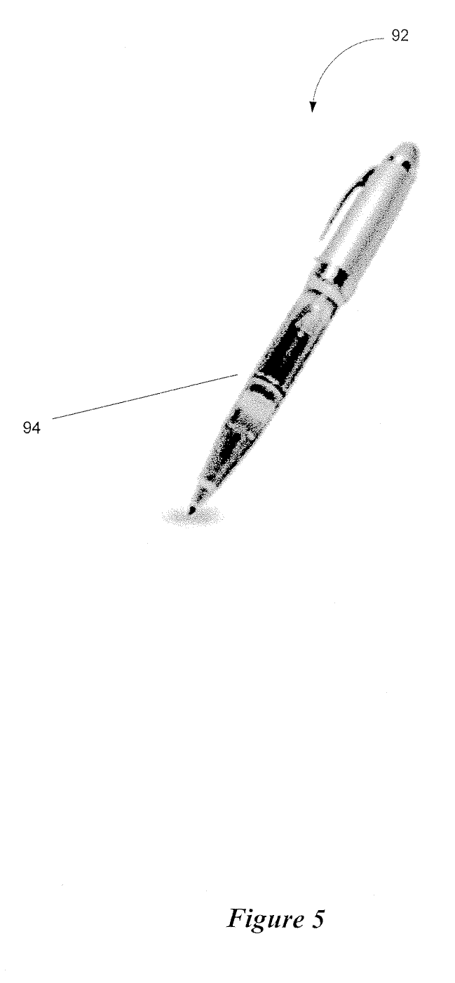

FIG. 5 shows a light pen 92 in one embodiment. The light pen displays a light at pen section 94, allowing a user to write at night without fully arousing to the hypnopompic or waking state. This allows the dream fragment to solidify before muscle movement and light cause the dream to dissipate. In one embodiment a light pen 92 comes in a kit distributed to cognitively intact individuals.

One embodiment may be used with non-intact individuals generally but not necessarily elderly. For a non-intact group, pockets of memory will be used to engage older, intact memories of the brain, linking the client reminiscence, capturing emotions associated with these events, and providing an opportunity to be successful in communication. This process adds to the client's self-esteem, sense of accomplishment, ego integrity, and feelings of group cohesiveness.

In one embodiment, training for a therapist will be included. It is preferable, but not necessary that any therapist has at least one, but preferably all of the following characteristics: compassion and love for the elderly; patience; intelligence; a good sense of humor; a good listener; some real world experience with counseling the aged; a good intuitive understanding of dreaming; a good intuitive understanding of reminiscing; a gentle rapport with clients; willingness to be creative and try new ways of thinking; interest in helping an elderly person to conduct a life review; and willing to keep a dream journal themselves.

One embodiment includes a new approach to geriatrics that requires interest in a broad range of topics. Dreaming, journaling, reminiscing and conversation are all combined to make an embodiment an effective approach. The primary drive in a good therapist is curiosity for what is inside the client. Our culture focuses so much on external realities that a therapy session is often the first time in a client's life that the focus is turned in on them. One embodiment is a process of discovery for the therapist and the client both. A properly trained therapist skilled in the art can instill confidence in the client. All interventions in one embodiment are gentle and non-coercive. The therapist is not guiding the process with a clear agenda. Rather the client sets the pace and trajectory of the therapy.

When selecting a patient there are several factors that promote the success of a therapy session. This list is not exhaustive but provides examples of several possible criterions. The criterion are: preferably, the client is relatively cognitively intact, that is they have good access to recent and remote memories; preferably, they are willing to journal their dreams; preferably, they are willing to share their memories with a therapist; preferably they are willing to work with a therapist on a continual basis, how frequently they meet can be mutually decided by the client and the therapist; preferably, the Client will have some means to pay for the services, a sliding scale is possible here. Preferably, the client is eager to self-explore and gain some insight into their unconscious and conscious functioning; and if the client has dementia it should not be too severe as to impede the process.

In an alternate embodiment, work can still be done with the client if there is a cognitive impairment. An embodiment is a combination of both dreams and reminiscing. Since memories often have a very dreamlike quality the therapy can proceed emphasizing the aspects of the mind that can still reminisce.

While the preferred embodiment of the invention has been illustrated and described, as noted above, many changes can be made without departing from the spirit and scope of the invention. For example, the method of dream therapy to facilitate life review may be appropriate anywhere a senior in a long-term care facility is lonely, needs companionship or wants to review their life. In other embodiments, the method may be employed to persons in activities, recreational therapy, social work or hospice care to facilitate preparation for death. Further, the method may be appropriate in alternate methods of counseling that seek to prepare an individual for acceptance and closure. Accordingly, the scope of the invention is not limited by the disclosure of the preferred embodiment. Instead, the invention should be determined entirely by reference to the claims that follow.

The embodiments of the invention in which an exclusive property or privilege is: claimed are defined as follows:

1. A method of dream therapy to facilitate life review, comprising:
recalling a dream fragment;
associating with the dream fragment;
amplifying the dream fragment;
reminiscing about life events; and
combining the amplified dream fragment and reminisced life events to conduct a life review.
